(19) United States
(12) Patent Application Publication
(10) Pub. No.: US 2019/0365649 A1
(43) Pub. Date: Dec. 5, 2019

(54) COMPOSITIONS COMPRISING LIPOSOME-ENCAPSULATED THIAZOLIDINEDIONES AND LIPOSOME-ENCAPSULATED VASODILATORS, AND THEIR USE TO AMELIORATE ATHEROMA

(71) Applicant: BOARD OF REGENTS OF THE UNIVERSITY OF TEXAS SYSTEM, Austin, TX (US)

(72) Inventors: David D. McPherson, Houston, TX (US); Shao-Ling Huang, Sugarland, TX (US); Melvin Klegerman, Houston, TX (US); Susan T. Laing, Houston, TX (US); Patrick Kee, Houston, TX (US); Tao Peng, Houston, TX (US); Hyunggun Kim, Hwasung (KR); Melanie Moody, Sugar Land, TX (US)

(73) Assignee: BOARD OF REGENTS OF THE UNIVERSITY OF TEXAS SYSTEM, Austin, TX (US)

(21) Appl. No.: 16/428,681

(22) Filed: May 31, 2019

Related U.S. Application Data
(60) Provisional application No. 62/678,869, filed on May 31, 2018.

(51) Int. Cl. A61K 9/127; A61K 31/506; A61K 47/54; A61K 47/06

(52) U.S. Cl. CPC A61K 9/127; A61K 31/506; A61K 33/00; A61K 47/06; A61K 47/543

(57) ABSTRACT

Disclosed are compositions comprising a first liposome, comprising at least one lipid selected from the group consisting of saturated phospholipids, unsaturated phospholipids, mixed phospholipids, and cholesterol, and at least one vasodilator; and a second liposome, comprising at least one lipid selected from the group consisting of saturated phospholipids, unsaturated phospholipids, mixed phospholipids, and cholesterol, at least one thiazolidinedione, and at least one compound having an affinity for at least one component of an atheroma. Also disclosed are methods for forming the compositions and methods of using the compositions to ameliorate atheroma.

### GOVERNMENT SUPPORT CLAUSE

This invention was made with government support under Grants No. HL-074002 and No. HL135092 awarded by the National Heart, Lung, and Blood Institute (NHLBI) of the United States National Institutes of Health (NIH). The government has certain rights in the invention.

### BACKGROUND OF THE INVENTION

The present disclosure relates generally to the field of amelioration of atheroma, particularly atheroma arising from stent implantation in an artery.

The implantation of stents in arteries is a well-established and efficacious technique in treating one or more disorders of the circulatory system. However, stents are foreign to the mammalian body, and as a result, the body may react against the implantation of such foreign body. The body's reactions may include atheroma formation and/or peri- and/or in-stent restenosis.

A number of strategies have been developed to reduce atheroma formation and/or in-stent restenosis. One strategy involves bioabsorbable stents, the dissolution of which may be promoted to reduce the duration of exposure of stent components to the arterial wall. This strategy has a drawback in that it cannot be used in therapies where long-term stent retention is desired. Another strategy is to deliver anti-proliferative drugs such as sirolimus and paclitaxel using drug-eluting stents to inhibit endothelial and smooth muscle cell proliferation. However, this strategy also has a limited time frame before the drug substantially completely elutes from the stent. Neither of these strategies stabilizes the atheroma that surrounds the stent (i.e., the peri-stent area). A third strategy is catheter-based therapeutic delivery at the time of intervention to overcome the limitations imposed by stent-based strategies. One example is the delivery of paclitaxel using drug-coated balloons in peripheral arterial interventions. A fourth strategy involves chronic administration of anticoagulants such as clopidogrel and aspirin (Alfonso 2014; Yahagi 2016). However, chronic administration of anticoagulants has potential side-effects that render it unsuitable for certain patient populations.

Accordingly, a need remains for treatment options to prevent or minimize atheroma formation and/or peri- and/or in-stent neoatherosclerosis.

### SUMMARY OF THE INVENTION

In one embodiment, the present disclosure relates to a composition, comprising a first liposome, comprising at least one lipid selected from the group consisting of saturated phospholipids, unsaturated phospholipids, mixed phospholipids, and cholesterol, and at least one vasodilator; and a second liposome, comprising at least one lipid selected from the group consisting of saturated phospholipids, unsaturated phospholipids, mixed phospholipids, and cholesterol; at least one thiazolidinedione, and at least one compound having an affinity for at least one component of an atheroma.

In one embodiment, the present disclosure relates to a method comprising administering a first liposome which comprises at least one lipid selected from the group consisting of saturated phospholipids, unsaturated phospholipids, mixed phospholipids, and cholesterol, and at least one vasodilator, into an artery in proximity to a stent; applying a first ultrasound treatment in proximity to the stent, whereby the at least one vasodilator is released by the first liposome; administering a second liposome which comprises at least one lipid selected from the group consisting of saturated phospholipids, unsaturated phospholipids, mixed phospholipids, and cholesterol, at least one thiazolidinedione, and at least one compound having an affinity for at least one component of an atheroma, into an artery of a mammal in proximity to a stent; and applying a second ultrasound treatment in proximity to the stent, whereby the at least one thiazolidinedione is absorbed by the atheroma.

In one embodiment, the present disclosure relates to a kit, comprising a composition referred to above and instructions for performing a method referred to above.

In one embodiment, the present disclosure relates to a method comprising forming a first liposome, comprising at least one lipid selected from the group consisting of saturated phospholipids, unsaturated phospholipids, mixed phospholipids, and cholesterol; introducing at least one vasodilator into the first liposome; combining at least one lipid selected from the group consisting of saturated phospholipids, unsaturated phospholipids, mixed phospholipids, and cholesterol, with at least one thiazolidinedione, in a solvent, to form a second liposome comprising the at least one lipid and the at least one thiazolidinedione; and conjugating at least one compound having an affinity for at least one component of an atheroma to the second liposome.

We have developed a therapeutic carrier and enhanced delivery strategy to stabilize atherosclerotic beds at the time of intervention (Beckman; Abdulhannan 2012; Lammer 2011; Huang 2009; Kee 2014), overcoming difficulties with present strategies, allowing stabilization of atheroma, and improving physiologic blood flow. Our approach may fulfill an unmet need in being an active intervention instead of a largely palliative strategy.

### BRIEF DESCRIPTION OF THE DRAWINGS

The disclosure may be understood by reference to the following description taken in conjunction with the accompanying drawings, in which like reference numerals identify like elements, and in which:

FIG. 1 shows delivery of pioglitazone into the arterial walls of swine by EKOS ultrasound, as described in Example 5.

FIG. 2A shows the effect of ultrasound on restenosis in swine, as described in Example 5.

FIG. 2B shows the effect of ultrasound on intima:media ratio in swine, as described in Example 5.

While the subject matter disclosed herein is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DESCRIPTION OF ILLUSTRATIVE EMBODIMENTS

Various illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

While the subject matter disclosed herein is susceptible to various modifications and alternative forms, specific embodiments thereof have been shown by way of example and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

The present subject matter will now be described. Various structures, systems, and devices are described for purposes of explanation only and so as to not obscure the present disclosure with details that are well known to those skilled in the art. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition will be expressly set forth in the specification in a definitional manner that directly and unequivocally provides the special definition for the term or phrase.

As therapeutic delivery carriers, liposomes are degradable and can be manipulated to incorporate physiologically active agents. They act as microreservoirs, prolonging regional content release (Unger 2001a; Unger 2001b), are small molecular structures, and the complexes can be made smaller by filtering or sonication. Liposomes are nontoxic targeted delivery systems and can carry either hydrophilic or hydrophobic compounds in the aqueous compartment or within the bilayers. We have developed an echogenic targetable liposomal complex (ELIP) that can incorporate air and bioactive or low-solubility gases such as perfluorocarbons. These liposomes are developed by formulation and composition such that during lyophilization, small amounts of gas are entrapped in multiple lipid bilayers or in the lumen, resulting in their echogenic properties. In contrast to standard gas-filled microbubbles that require much of the microbubble volume be made up of gas to be acoustically active, our ELIPs have the capacity to also carry large therapeutic payloads (Kim 1987). During manufacturing, we maintain targeting capabilities and echogenicity properties, while encapsulating therapeutic payloads.

We have had great success developing ultrasound-triggered therapeutic release strategies from ELIP. As an example, we developed an ex-vivo porcine carotid model to assess the vascular effects of ultrasound-mediated delivery of a bioactive gas, nitric oxide (NO), from NO-ELIP (Bekeredjian 2003). In an ex-vivo physiologic setting, NO-ELIP were infused into the lumen of carotid arteries, and exposed to 1-MHz pulsed ultrasound at a peak rarefactional pressure amplitude of 0.17 MPa, while acoustic cavitation emissions were monitored. Changes in vascular tone were compared. Our results demonstrate that ultrasound-triggered NO release from NO-ELIP induces potent vasorelaxation and is concomitant with stable cavitation (ibid.). Based upon these and other data, we propose that the interaction of ultrasound with NO-ELIP can enhance therapeutic delivery to vascular tissue.

Acoustic cavitation has been shown to mediate many therapeutic ultrasound applications, including drug and gene release and delivery (Kodama 2006; Newman 2007; Lawrie 2003; Unger 2001a; Unger 2001b; Bekeredjian 2003; Chen 2003; Suzuki 2007; Hernot 2008; Suzuki 2010; Chen 2012; Phillips 2012; Hitchcock 2010), and sonothrombolysis (Hitchcock 2010; Everbach 2000; Datta 2006; Prokop 2007; Datta 2008; Hitchcock 2011). Passive and active cavitation detection techniques have been developed to monitor acoustic cavitation (Roy 1990; Mandanshetty 1991). Passive schemes employ a transducer that listens passively (without transmitting) to emissions from acoustically activated microbubbles. Multiple-element arrays have been developed to allow for spatial resolution of bubble activity over a large area (Farny 2009; Gyongy 2010; Salgaonkar 2009). Active subharmonic imaging techniques for bubble detection using ultrasound arrays have been implemented (Shankar 1998; Krishna 1999; Shi 1999). Passive cavitation imaging (PCI) uses a modified delay-and-sum algorithm to beamform cavitation emissions. Image resolution depends only on the frequency-dependent diffraction pattern of the array and is independent of the therapeutic insonification pulse duration and waveform shape, enabling the technique to be applied to both continuous-wave and pulsed ultrasound exposures (Haworth 2012). For typical diagnostic imaging arrays, the diffraction pattern is on the order of one wavelength in the lateral dimension and tens of wavelengths in the axial direction (Salgaonkar 2009; Haworth 2012). The cavitation activity is temporally localized based on the PCI frame rate, which can be as high as 8 kHz using the Vantage Verasonics system. PCI acquisition is synchronized with the therapeutic transmit pulse repetition frequency (PRF), providing independent cavitation measurements for each transmitted pulse (Haworth 2012; Haworth 2015). The 250-kHz pulsed ultrasound exposure scheme that we propose to treat arteries utilizes low amplitude and low frequency pulses. This strategy will be initially developed for coronary therapeutic delivery but can be translated to peripheral arteries if found to have superior efficacy.

Ultrasound-mediated therapeutic release into coronary and peripheral arteries is challenging. Insonification of the arteries is necessary for carrier-based therapeutic delivery. We have developed both external and internal ultrasonic arrays/catheters for ultrasonic mediated therapeutic release from our carriers.

Thiazolidinediones, such as rosiglitazone and pioglitazone, tend to function as PPARγ agonists and have local anti-inflammatory effects in the arterial wall (Buchanan 2010). PPARγ agonists target genes relevant to smooth muscle cell proliferation and macrophage activation, leading to modulation of atheroma development (Cheng 2005; Coussios 2004). Systemic effects have limited clinical utility (Gruber 2014; Haworth 2013). To avoid these complications, local delivery to atheroma may be warranted. We developed novel ELIP for rosiglitazone (RGN) encapsulation and release (RGN-ELIP) (Moody 2008). 91% rosiglitazone can be loaded with retention of echogenicity. Encapsulated rosiglitazone demonstrated local release and ability to inhibit proliferation of vascular smooth muscle cells (VSMC) with anti-inflammatory effect (Ibid.).

We have developed a carrier platform (echogenic liposomes) for vascular stabilization (Huang 2004). This carrier can encapsulate nitric oxide for vascular dilation and enhanced permeability (Huang 2008; Huang 2006a; Huang 2006b). It can also encapsulate a therapeutic (e.g., pioglitazone) for stabilization of inflammation and be targeted to an atheroma marker such as an adhesion molecule or fibrin. The two together can dilate the peripheral or coronary artery at the time of stent placement, bathe the persistent area and stabilize the acute inflammation caused by the stent procedure, minimizing stent neoatherogenesis (Moody 2008; Moody 2008; Moody 2017). Novel ultrasound activation (via an intra-arterial ultrasound catheter) of the ELIP carriers will release the carrier contents only in the pertinent area, with maximal therapeutic delivery to the area (Moody 2017; Klegerman 2010). Altogether, by decreasing persistent inflammation, promoting re-endothelialization and inhibiting neointimal hyperplasia, we expect to stabilize the arterial bed (peripheral or coronary) at the time of intervention, thereby preventing or minimizing peri- and in-stent restenosis and potentially negating long term anti-platelet therapy.

In one or more embodiments, we disclose a biodegradable carrier that can be injected intravenously or intra-arterially to the target site; echogenic immunoliposomes (ELIP); a targeting moiety that can attach the carrier to the site of action; encapsulation of a vasodilator (i.e., nitric oxide) and an anti-inflammatory (i.e. pioglitazone) in separate, but simultaneously or sequentially administered, carriers, allowing delivery of the therapeutics to the target site; use of an ultrasonic catheter to open the therapeutic carrier and deliver its contents to the site of action; and stabilization of an implanted stent and/or a persistent atheroma by reducing inflammation promoting reendothelialization and inhibiting neointimal hyperplasia with the potential to minimize/negate the need for long term antiplatelet therapies.

In one embodiment, the present disclosure relates to a composition, comprising: a first liposome, comprising at least one lipid selected from the group consisting of saturated phospholipids, unsaturated phospholipids, mixed phospholipids, and cholesterol, and at least one vasodilator; and a second liposome, comprising at least one lipid selected from the group consisting of saturated phospholipids, unsaturated phospholipids, mixed phospholipids, and cholesterol; at least one thiazolidinedione; and at least one compound having an affinity for at least one component of an atheroma.

As stated above, the composition also comprises a first liposome. The first liposome comprises at least one lipid selected from the group consisting of saturated phospholipids, unsaturated phospholipids, mixed phospholipids, and cholesterol. The lipid formulation of the first liposome may be identical to that of the second liposome, or may be different. In one embodiment, the first liposome comprises egg PC, dipalmitoylphosphatidylcholine (DPPC), DPPE-PEG2000, DPPG, and cholesterol. In a further embodiment, the first liposome comprises 27 mole % egg PC; 42 mole % DPPC; 8 mole % DPPE-PEG2000; 8 mole % DPPG; and 15 mole % cholesterol.

The first liposome also comprises at least one vasodilator. In one embodiment, the at least one vasodilator is nitric oxide (NO).

As stated above, the first liposome comprises at least one lipid and at least one vasodilator. The at least one lipid generally forms a lipid monolayer, bilayer, etc. that defines a liposome exterior and a liposome interior. The at least one vasodilator may be present as a solute in the liposome interior and/or as a dispersant within the lipid layer(s).

As stated above, the composition also comprises a second liposome. The second liposome comprises at least one lipid selected from the group consisting of saturated phospholipids, unsaturated phospholipids, mixed phospholipids, and cholesterol. In one embodiment, the second liposome comprises at least one lipid selected from the group consisting of 1,2-distearoyl-sn-glycero-3-phosphocholine (DSPC); 1,2-dioleoyl-sn-glycero-3-phosphoethanolamine-N-[4-(p-maleimidophenyl)butyramide] (MPB-DOPE); 1,2-dioleoyl-sn-glycero-3-phosphocholine (DOPC); dipalmitoylphosphatidylglycerol (DPPG), and cholesterol. The inclusion of MPB-DOPE in the second liposome may allow convenient thiol linkage of the at least one compound having an affinity for at least one component of an atheroma to the main body of the liposome. In a further embodiment, the second liposome comprises 52 molar % DSPC, 8 molar % MPB-DOPE, 30 molar % DOPC, and 10 molar % cholesterol.

Alternatively or in addition, the first liposome, the second liposome, or both may further comprise an octofluoroalkane, such as octofluoropropane or octofluoropentane (OFP). In one embodiment, the octofluoroalkane is OFP. Though not to be bound by theory, OFP (or other octofluoroalkane) may increase the visibility of a liposome during ultrasound imaging; increase the rate of disruption of the liposome upon the application of ultrasound energy; increase the completeness of disruption of the liposome upon the application of ultrasound energy; or two or more thereof; all relative to an otherwise identical liposome lacking OFP.

As stated above, the second liposome comprises at least one thiazolidinedione. A “thiazolidinedione” as used herein refers to an organic compound having at least one moiety with the following structure:

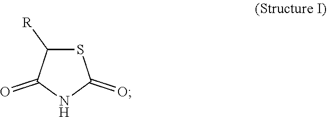

wherein R is an organic moiety. The thiazolidinedione may also comprise a pharmaceutically-acceptable salt of a compound having Structure I. Thiazolidinediones are known and at least some thiazolidinediones are approved by regulatory agencies in at least some countries for treatment of diabetes mellitus type 2. Evidence points to thiazolidinediones as also stabilizing atherosclerotic plaques and atheromas in arteries.

In one embodiment, the at least one thiazolidinedione is selected from the group consisting of pioglitazone, rosiglitazone, lobeglitazone, ciglitazone, darglitazone, englitazone, netoglitazone, rivoglitazone, troglitazone, and balaglitazone. In a further embodiment, the at least one thiazolidinedione is selected from the group consisting of rosiglitazone and pioglitazone. In yet a further embodiment, the at least one thiazolidinedione is pioglitazone.

As stated above, the second liposome also comprises at least one compound having an affinity for at least one component of an atheroma. The person of ordinary skill in the art is aware that atheromas typically comprise macrophage cells, lipids, calcium, adhesins, and fibrous connective tissue, the later including but not limited to fibrin. The person of ordinary skill in the art is also aware that the placement of a stent in an artery tends to encourage atheroma formation in the artery in proximity to the stent.

In one embodiment, the at least one compound having an affinity for at least one component of an atheroma is selected from the group consisting of fibrin binding peptides (FBPs) and antibodies to at least one intercellular adhesion molecule (anti-ICAMs). In a particular embodiment, the at least one compound having an affinity for at least one component of an atheroma is an FBP having the amino acid sequence Gly-Pro-Arg-Pro-Pro-Gly-Gly-Gly-Cys (GPRPPGGGC) (SEQ ID NO:1).

In embodiments wherein the at least one compound having an affinity for at least one component of an atheroma is an FBP having SEQ ID NO:1 and the second liposome comprises MBP-DOPE, the MPB-DOPE in the second liposome may be conjugated to the C-terminal cysteinyl thiol group through a thioether linkage. A triglycyl spacer maintains a spacing between the cysteine and the fibrin-binding pentapeptide formed by the N-terminal Gly-Pro-Arg-Pro-Pro subsequence.

As stated above, the second liposome comprises the at least one lipid, the at least one thiazolidinedione, and the at least one compound having an affinity for at least one component of an atheroma. The at least one lipid generally forms a lipid monolayer, bilayer, etc. that defines a liposome exterior and a liposome interior. The at least one thiazolidinedione may be present as a solute in the liposome interior and/or as a dispersant within the lipid layer(s). The at least one compound having an affinity for at least one component of an atheroma may be present on the liposome exterior, such as by a covalent bond to a lipid, e.g., the thioether linkage to MBP-DOPE described above.

In one embodiment, the second liposome comprises egg PC, DPPC, 1,2-dipalmitoyl-sn-glycero-3-phosphoethanolamine-N-[methoxy(polyethyleneglycol)-2000] (DPPE-PEG2000), DPPG, and cholesterol in a ratio of 27:42:8:8:15 molar percent; rosiglitazone complexed to hydroxypropyl-beta-cyclodextrin (HPβCD); and thiolated enlimomab MAb (anti-ICAM-1) as the at least one compound having an affinity for at least one component of an atheroma.

The compositions may be formed by any techniques known to the person of ordinary skill in the art. In one embodiment, the present disclosure relates to a method, comprising forming a first liposome, comprising at least one lipid selected from the group consisting of saturated phospholipids, unsaturated phospholipids, mixed phospholipids, and cholesterol; introducing at least one vasodilator into the first liposome; combining at least one lipid selected from the group consisting of saturated phospholipids, unsaturated phospholipids, mixed phospholipids, and cholesterol, with at least one thiazolidinedione, in a solvent, to form a second liposome comprising the at least one lipid and the at least one thiazolidinedione; and conjugating at least one compound having an affinity for at least one component of an atheroma to the second liposome.

The first liposome may be formed by combining at least one lipid selected from the group consisting of saturated phospholipids, unsaturated phospholipids, mixed phospholipids, and cholesterol in a solvent, such as chloroform or ethanol. In one embodiment, the solvent is selected from the group consisting of chloroform and ethanol. In a further embodiment, the solvent is ethanol. In another further embodiment, the solvent is chloroform.

In one embodiment, a first liposome comprising egg PC, DPPC, DPPE-PEG2000, DPPG, and cholesterol may be formed by mixing the lipids in the solvent. The solvent is then removed by evaporation under an inert gas, e.g., argon, while the solution is agitated at 50° C. The lipid film resulting from solvent evaporation is then placed under vacuum for 4-6 hr for complete removal of the solvent. The dry lipid film is rehydrated with deionized water and sonicated in a water bath for 5 min. An equal volume of 0.32 M mannitol is then added. The mixture is then frozen on dry ice and lyophilized for 24 hr. After lyophilization, the dry cake of first liposome is topped with OFP gas.

The at least one vasodilator may be introduced into the first liposome by any appropriate technique. In one embodiment, wherein the at least one vasodilator is NO, the NO may be introduced into a lyophilized first liposome by reconstituting the first liposome with NO/octafluoropentane in a 1:9 ratio dissolved in water.

In the combining to form the second liposome, the at least one lipid and the at least one thiazolidinedione may be combined in any solvent in which both the at least one lipid and the thiazolidinedione are soluble. In one embodiment, the solvent is selected from the group consisting of chloroform and ethanol. In a further embodiment, the solvent is ethanol. In another further embodiment, the solvent is chloroform.

In a particular embodiment, the second liposome comprising the at least one lipid and the at least one thiazolidinedione may be formed as follows. The lipids DSPC, MBP-DOPE, DOPC, DPPG, and cholesterol, and the at least one thiazolidinedione pioglitazone (as pioglitazone-HCl) are mixed in the solvent. The solvent is then removed by evaporation under an inert gas, e.g., argon, while the solution is agitated at 50° C. The lipid film resulting from solvent evaporation is then placed under vacuum for 12-16 hr for complete removal of the solvent. The dry lipid film is rehydrated with deionized water containing 0.32 M mannitol at 10 mg of lipid/ml. The hydrated lipid is then incubated at 55° C. for 30 min to insure all lipids are in the liquid crystalline phase during hydration. The mixture is then frozen on dry ice and lyophilized for 48 hr. After lyophilization, the dry cake of second liposome is topped with OFP gas. Prior to use, the second liposome is reconstituted with water.

In another particular embodiment, the second liposome comprising the at least one lipid and the at least one thiazolidinedione may be formed as follows. A 5 mg combination of the lipids DSPC (52 molar %), MBP-DOPE (30 molar %), DOPC (8 molar %), and cholesterol (10 molar %) is dissolved in ethanol and the mixture is placed in a 250 ml round bottom flask. The solvent is evaporated by slowly rotating the flask under a gentle argon stream in a 56° C. water bath in chemical fume hood. Pioglitazone (1-2 mg) is dissolved in 1 ml ethanol. The formed lipid film is then re-dissolved with the pioglitazone ethanol solution (1 ml). The resultant ethanol solution is then injected into 3 ml of 0.32 M mannitol through a 31-gauge needle under stirring. The resultant liposomes are put in an evaporator under 75° C. under vacuum to remove ethanol. The pioglitazone-loaded ELIP is separated from free pioglitazone by centrifugation at 5,800 g for 10 min. The pioglitazone-loaded ELIP (PGN-ELIP) is subjected to antibody conjugation. The conjugated PGN-ELIP is frozen on dry ice (−80° C.) for 4 hours. The frozen sample was then lyophilized for 48 hrs. After lyophilization, the dry cake is topped with octafluoropropane gas and capped with a butyl rubber septum.

Regardless how the second liposome is formed, at least one compound having an affinity for at least one component of an atheroma is conjugated to the second liposome. In one embodiment, wherein the second liposome comprises MBP-DOPE and the at least one compound having an affinity for at least one component of an atheroma comprises the FBP having SEQ ID NO:1, the FBP having SEQ ID NO:1 may be conjugated to the reconstituted second liposome by mixing together in water or a phosphate buffer of pH 6.6-6.7 at room temperature for 12-16 hr. After mixing, the conjugated FBP having SEQ ID NO:1 may be removed from solution by centrifugation and washing with 0.32M mannitol or a phosphate-buffered saline (not necessarily isotonic).

In another embodiment, the present disclosure relates to methods of ameliorating atheroma in a patient in whose body a stent has been implanted in an artery. In one embodiment, the present disclosure relates to a method, comprising administering a first liposome which comprises at least one lipid selected from the group consisting of saturated phospholipids, unsaturated phospholipids, mixed phospholipids, and cholesterol, and at least one vasodilator, into the artery in proximity to the stent; applying a first ultrasound treatment in proximity to the stent, whereby the at least one vasodilator is released by the first liposome; administering a second liposome which comprises at least one lipid selected from the group consisting of saturated phospholipids, unsaturated phospholipids, mixed phospholipids, and cholesterol, at least one thiazolidinedione, and at least one compound having an affinity for at least one component of an atheroma, into an artery of a mammal in proximity to a stent; and applying a second ultrasound treatment in proximity to the stent, whereby the at least one thiazolidinedione is absorbed by the atheroma;

The first and second liposomes, the at least one lipid, the at least thiazolidinedione, the at least one compound having an affinity for at least one component of an atheroma, and the at least one vasodilator may be as described above and the liposomes may be formed as described above.

The stent may be either a stent of bare material, e.g., a metal, or a stent coated with one or more bioactive molecules. In one embodiment, stent is bare metal, i.e., is metal uncoated by any bioactive molecule.

The stent may have been implanted in any artery. In one embodiment, the artery is selected from the group consisting of a carotid artery, a peripheral artery, and a coronary artery. In one further embodiment, the artery is a carotid artery. In another further embodiment, the artery is a coronary artery. In yet another further embodiment, the artery is a peripheral artery.

Though not to be bound by theory, the at least one thiazolidinedione may be absorbed by the atheroma in one or more ways. For example, the second ultrasound treatment may promote release of the at least one thiazolidinedione by the second liposome and subsequently promote absorption of the released at least one thiazolidinedione by the atheroma. For another example, the second ultrasound treatment may force the second liposome into the atheroma and the arterial wall, from where the second liposome may be internalized by cells, which then dismantle the second liposome and intracellularly release the at least one thiazolidinedione.

The second liposome and the first liposome may be administered in either order or simultaneously. In one embodiment, the administering the first liposome (the liposome carrying the at least one vasodilator) is performed prior to the applying the first ultrasound treatment; and the applying the first ultrasound treatment is performed prior to the administering the second liposome (the liposome carrying the at least one thiazolidinedione). In other words, the at least one vasodilator may be released to the artery in proximity to the stent prior to releasing the at least one thiazolidinedione in proximity to atheroma. By doing so, though not to be bound by theory, the at least one vasodilator may dilate the artery, and the at least one thiazolidinedione may ameliorate the atheroma.

The first ultrasound treatment and the second ultrasound treatment may be applied in proximity to the stent from any ultrasound source. In one embodiment, the first ultrasound treatment and/or the second ultrasound treatment may be applied from an intra-arterial device, which may be the same device that delivered the first liposome and the second liposome to the artery in proximity to the stent. In another embodiment, the first ultrasound treatment and/or the second ultrasound treatment may be applied from a device deployed externally to the patient's body. In other embodiments, different devices may be used to deliver the first ultrasound treatment and the second ultrasound treatment.

The parameters of the first ultrasound treatment and the second ultrasound treatment may be selectively chosen to provide optimal unloading of the at least one vasodilator from the first liposome and optimal absorption of the at least one thiazolidinedione by the atheroma. In one embodiment, the first ultrasound treatment has the following parameters: power 9 W, pulse duration 15:00/30:00 ms, and frequency 2,200 kHz. Alternatively or in addition, in one embodiment, the second ultrasound treatment has the following parameters: power 9 W, pulse duration 15:00/30:00 ms, and frequency 2,200 kHz. However, in other embodiments, the parameters of the first ultrasound treatment and the second ultrasound treatment may vary, as will be apparent to the person of ordinary skill in the art having the benefit of the present disclosure.

Administration of the first and second liposomes and application of the first and second ultrasound treatments may be performed by use of an intraarterial ultrasound catheter, such as catheters marketed by EKOS, such as the EndoSonic® catheter.

Though not to be bound by theory, performing the method may ameliorate atheroma in proximity to an implanted stent by at least one of reducing inflammation or promoting reendothelialization in an artery in proximity to the implanted stent.

In one embodiment, the present disclosure relates to a kit, comprising a composition as described above and instructions for performing a method of ameliorating atheroma in a patient in whose body a stent has been implanted in an artery, also as described above.

The following examples are included to demonstrate particular embodiments of the disclosure. It should be appreciated by those of skill in the art that the techniques disclosed in the examples which follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the spirit and scope of the disclosure.

### EXAMPLES

### Example 1. First Liposomes Comprising NO and Second Liposomes Comprising Pioglitazone

Introduction

In the management of atherosclerotic lesions, stent implantation is effective against acute luminal loss but has potential of late loss due to peri- and/or in-stent restenosis (Abdulhannan 2012). Neointimal growth and peri- and/or in-stent restenosis are the results of acute arterial injury by angioplasty and/or platelet- and/or leukocyte-activation due to stent component exposure and smooth muscle cell migration (Farb 2002; Brower 1996). Stents delivering anti-proliferative agents such as sirolimus and paclitaxel are effective against neointimal proliferation but the unpredictable risk of very late stent thrombosis due to impaired re-endothelialization, delayed vascular healing, and hypersensitivity reaction to stent components, polymer coating, and the drug remains a significant problem (Finn 2007; Nakazawa 2009). Despite the success of drug-eluting stents in reducing peri- and/or in-stent restenosis in certain coronary lesions, the use of drug eluting stent in peripheral artery disease has reported disappointing long-term results (Duda 2005; Duda 2002).

These stent-related complications lead to a re-visit of other strategies for local delivery of anti-proliferative or “pro-healing” drug without the need of an implanted drug delivery system. The potential benefits of such delivery strategy include drug delivery at therapeutic doses upfront without the restriction imposed by stent-based delivery systems, the absence of any stent components that could trigger neoinitimal hyperplasia, and the feasibility of delivering therapeutics to sites that may not be suited for stent implantation such as small vessels, bifurcation lesions, ostial lesions, saphenous vein grafts, arterial-venous fistulas, and femoral-popliteal arteries.

We have demonstrated that local delivery of therapeutics could acutely stabilize atheroma and result in durable anti-inflammatory effects against neointimal hyperplasia (Huang 2009). Our delivery platform is based on an echogenic liposomal formulation with the capabilities to target via surface functionalization, encapsulate a host of payload in its various compartments, and respond to ultrasound activation for controlled payload release. Our previous studies have demonstrated the versatility of such delivery platform in delivering bioactive gases and other therapeutics for molecular imaging of atheroma (Kee 2014; Kim 2013; Kim 2010), modulating the development of atheroma (Huang 2009), enhancing the effects of thrombolytics (Laing 2012), and reducing the infarct size in stroke (Kim 2014; Peng 2013).

In this study, we employed a novel endovascular ultrasound system to enable site-specific delivery of therapeutics from echogenic liposomes into stented peripheral arteries. The echogenic liposomes were formulated to target adhesion molecule expression in the vicinity of the stented vessels and deliver therapeutic doses of nitric oxide for acute anti-oxidative and anti-platelet effects and pioglitazone for sustained anti-inflammatory and anti-proliferative effects. We hypothesized that such ultrasound delivery strategy of echogenic liposomal payload would reduce inflammation, promote reendothelialization, and inhibit neointimal hyperplasia in the stented peripheral arteries in a large animal model of atherosclerosis.

Methods

Preparation and Characterization of NO-ELIP and Pioglitazone FLIP:

The preparation of ELIP, anti-ICAM-antibody-conjugated ELIP, nitric oxide-loaded ELIP and pioglitazone-loaded ELIP were described previously (Hamilton 2002a; Huang 2009; Lanza 2000; Kee 2014, Moody 2008, Huang 2014 Int J Nanomedicine submitted).

Preparation of Antibody-Conjugated ELIP:

For antibody conjugation prior to drug loading, ELIP were prepared as described above, and 1,2-dipalmitoyl-sn-glycero-3-phosphoethanolamine-N-(4-(p-maleimidophenyl) butyrate) (MPB-PE; Avanti Polar Lipids) was substituted for PE. For protein thiolation, 0.4 mg monoclonal anti-human/porcine ICAM-1 (Clone R6.5; eBioscience, Thermofisher, Waltham, Mass.) and 1.6 mg nonspecific mouse Immunoglobulin G (IgG; Rockland Immunochem., Inc., Gilbertsville, Pa.) were reacted with 3-(2-pyridyldithiolpropionic acid)-N-hydroxysuccinimide ester (SPDP) at a SPDP:IgG protein molar ratio of 15:1 for 30 minutes at 24±1° C. Protein was separated from unreacted SPDP by gel chromatography on a 50 ml Sephadex G-50 column (Sigma-Aldrich, St. Louis, Mo., USA) equilibrated with 0.05M citrate-phosphate buffer at a pH of 5.5. Protein fractions were identified using a spectrophotometric technique (Genesys 10 uv, Thermo Electron Corp., Milford, Mass.) at a wavelength of 280 nm, pooled and concentrated to ˜2 ml using Centricon YM-10 centrifugal filter units (Millipore, Billerica, Mass., USA). The SPDP-activated protein was reduced in 25 mM dithiothreitol (DTT) for 30 minutes at 24±1° C. The thiolated protein was isolated using a Sephadex G-50 column, equilibrated, and eluted with pH 6.7 citrate-phosphate buffer. The thiolated protein was reacted with reconstituted MPB-ELIP (10 mg lipid/ml 0.1M phosphate buffer at a pH of 6.62) under argon overnight at 24±1° C. AbELIP were separated from free protein and low molecular weight products by gel filtration on a 20-ml Sepharose CL-4B column (Sigma-Aldrich) that had been pre-saturated with unconjugated, unlabeled ELIP according to an established method (Lasche 2003) and eluted with 0.02 M phosphate-buffered saline (PBS), pH 7.4. Liposome-containing fractions were identified by optical absorbance at a wavelength of 280 nm prior to elution of free IgG.

The conjugation efficiency (CE) of three lots of anti-ICAM-1-ELIP subsequently loaded with pioglitazone and used for the experiments in this study, as determined by a quantitative immunoblot assay (Klegerman 2002) for liposomal IgG ranged from 0.4 to 1.8 μg antibody/mg lipid (20% of total IgG conjugated) and 137 to 422 molecules antibody/liposome, respectively. Beckman Coulter Multisizer 4 analysis enabled determination of both ELIP number and size characteristics for ELIP >0.4 μm equivalent spherical diameter, which ranged from 506 to 661 nm; ELIP number ranged from 4.26×10⁸ to 1.23×10¹⁰ liposomes/mg lipid.

Swine Procedure:

The animal protocol was approved by the Animal Welfare Committee at the University of Texas Health Science at Houston. Atherosclerotic lesions were induced in both subclavian arteries and the right common carotid artery in three female Yucatan miniswines (weight=20 kg) using a combination of hyperlipidemic diet and balloon denudation. Two weeks prior to balloon denudation, the animals were fed a hyperlipidemic diet containing 2% cholesterol and 15% lard. Oral aspirin was commenced four days prior to balloon denudation. On the day of balloon denudation, animals were sedated with teletamine/zolazepam IM (4 mg/kg), intubated and ventilated, and anesthetized with inhaled isoflurane (1.5-3%). An incision was made in the right inguinal region and the right femoral artery was exposed for insertion of a 6F arterial sheath. Under fluoroscopic guidance, a 4F Fogarty over-the-wire embolectomy catheter was introduced to both subclavian arteries and right carotid arteries and an arterial segment of about 5 cm was denuded three times with the inflated balloon catheter. A total of nine arteries were denuded. The left carotid artery was not manipulated. The animals then continued on aspirin and the hyperlipidemic diet. Analgesia in the form of Buprenorphine IM (0.05 mg/kg) was given 2 to 4 times daily for up to a week after each survival procedure. Six to eight weeks after balloon denudation, the animals were sedated and anesthetized and subjected to balloon angioplasty and stent implantation in the denuded segments. An 8F arterial sheath was inserted into the left femoral artery. Under fluoroscopic guidance, a 3.2F intravascular ultrasound catheter (Volcano Corp) was advanced into both subclavian arteries and right carotid artery to visualize the lumen and arterial wall of the denuded segments. Bare metal stents (Boston Scientific, MN) measuring 6 mm×18 mm were deployed in the seven denuded arterial segments. Immediately after stent implantation, an EKOS catheter was then advanced with the catheter tip placed just proximal to the stent and the ultrasound element within the stented segment. Therapeutic agents were administered via the catheter. Therapeutic ultrasound was delivered by the EKOS EndoSonic® catheter using the following parameters: Power 9 W, pulse duration 15:00/30:00 ms, frequency 2,200 kHz. Arteries were randomized to receive treatments with NO-ELIP+anti-ICAM-1-pioglitazone-ELIP with and without ultrasound activation. Eight weeks later, a laparotomy was performed and an arterial sheath was inserted into the abdominal aorta. Under fluoroscopic guidance, IVUS catheter was inserted into both subclavian and right carotid arteries to visualize the changes in the luminal size and arterial wall thickness. After IVUS imaging, the animals were euthanized and the arterial segments were pressure perfused and fixed with formalin in situ and harvested for histological analysis.

To demonstrate payload delivery into the denuded arterial segments, an additional pig was subjected to similar procedures described above without the stent implantation. The lipid components of the ELIP were labeled with rhodamine and pioglitazone was labeled with FITC. Six to eight weeks after balloon denudation, the fluorescently labeled ELIPs were delivered via an EKOS catheter with ultrasound activation into the right carotid and right subclavian arteries and without ultrasound activation into the left subclavian artery. Thirty minutes after ELIP delivery, the animals were euthanized and the treated arterial segments were harvested for fluorescence microscopy.

Histological Analysis:

Arterial segments containing the stented tissue and several mm on either end were harvested and sent to a pathology laboratory (Alizee Pathology, Thurmont, Md.) for processing. Briefly: Specimens were processed through 8 stations of graded alcohols and xylene under a 2 hour per station program. Specimens were then infiltrated under the following schedule: a) 2-4 hour changes of acetone b) 48 hour change in Spurr/acetone, 50/50 concentration c) 48 hour change in Spurr 1 d) 48 hour change in Spurr 2. Specimens were then embedded in the Spurr 2 solution and placed in a 60° C. oven for 48 hours to polymerize. Sections were cut at 5 microns. Two sections each were cut from the proximal, middle, and distal regions within the stented portion of each artery. In addition, when enough tissue remained, two sections were cut from the proximal and distal regions just outside of the stented region as references. For each region, one section was stained with H&E and one stained with Movat's for histological evaluation. To measure the amount of neo-intimal hyperplasia, intima:media ratio was measured, the outer boundary of the media (area A), the interface between the intima and the media (area B) and the lumen (area C) were manually traced with NIH ImageJ software (Bethesda, Mass.) in each cross section of the arteries. The intimal volume (area B−area C) and the medial volume (area A−intimal volume) were calculated and expressed as intimal: medial ratio to correct for the size and shape of various arterial segments.

Image Analysis:

IVUS signal data were utilized to reconstruct acoustic intensity data sets in the polar coordinate (radial vs. circumferential axes) (Kim 2010). IVUS signal envelope data were collected at 1,024 data points per scan line. A total of 256 scan lines were collected along the radial direction per IVUS slice (Eagle Eye Gold catheter, Volcano Corporation, San Diego, Calif.). The acoustic intensity data sets in the polar coordinate were transformed to the Cartesian coordinate system for standard IVUS imaging (Kim 2010). As the imaged arterial segments of interest were relatively straight, it was assumed that the pullback direction of IVUS catheter was parallel to the longitudinal direction of the artery. Our graphical user interface (GUI)-based image processing system was utilized to interactively trace and segment the arterial structure for image analysis (Kim 2013; Kim 2010). In a blinded manner, borders of the endothelium/atheroma and the outer edge of the dense adventitia were manually segmented in each IVUS slice, and the acoustic enhancement within these arterial wall borders after each treatment was quantitated by both mean gray scale values (i.e. pixelated brightness data) and radio-frequency (RF) magnitude values (i.e. signal intensity data).

Statistical Analysis:

SigmaStat (Systat Software Inc., San Jose, Calif.) was utilized for statistical analyses. Student t-test was used to compare the differences in neointimal volume and intima-media ratio between treatments with and without ultrasound activation. Data were shown as mean and standard error of means. A p-value <0.05 was considered significant.

Results

Delivery of Fluorescently Labeled ELIPs into the Arterial Wall:

To demonstrate the delivery of the anti-ICAM-1-PGN-ELIP into the arterial wall, the lipid components of the ELIP were labeled with rhodamine and pioglitazone with FITC. In the right carotid and right subclavian arteries, ultrasound facilitated the delivery anti-ICAM-1-PGN-ELIP into the arterial wall. In the absence of ultrasound, the left subclavian artery treated with anti-ICAM-1-PGN-ELIP resulted in negligible delivery of the fluorescent payload into the arterial wall.

Intravascular Imaging:

Stented arteries exposed to ultrasound-facilitated ELIP payload delivery resulted in marked reduction in neointimal volume. Untreated 7.31±2.32 mm². Treated 1.24±0.25 mm².

Histological Analysis:

Intimal-medial ratio. Untreated 46.71±3.52. Treated 29.76±2.56.

Discussion

By employing a number of innovative strategies including site-specific, ultrasound-tuned, direct payload delivery, active molecular targeting, and combined delivery of short and long-acting anti-inflammatory agents via echogenic liposomes, we were able to reduce in-stent restenosis in a large animal model of atherosclerosis.

A number of strategies are currently developed to reduce peri- and/or in-stent restenosis. One way is to promote the dissolution of bioabsorbable stents and reduce the duration of exposure of stent components to the arterial wall. The other way is to deliver anti-proliferative drugs such as sirolimus and paclitaxel using drug-eluting stents to inhibit endothelial and smooth muscle cell proliferation. There is also a renewed interest in catheter-based therapeutic delivery at the time of intervention to overcome the limitations imposed by stent-based strategies. One example is the delivery of paclitaxel using drug-coated balloons in peripheral arterial interventions. Our approach using ultrasound-activatable ELIPs aims to improve the precision and flexibility of drug delivery at the target site and may offer a number of potential advantages. First, the amount of drug can be modulated with minimal drug loss during transit. Second, ultrasound activation can deliver precise amount of drug rather than relying on passive transfer as in the setting of drug-coated balloons. Third, a combination of therapeutic agents can be delivered concurrently, as in this example, nitric oxide for enhanced therapeutic delivery to all of the peri-stent area and pioglitazone (PGN) to stabilize the anti-inflammatory response associated with foreign body (stent) placement, to maximize both short- and long-term clinical outcomes. Finally, active targeting by means of antibody homing improves the retention and proximity of ELIPs at the target and improves the efficiency of ultrasound-facilitated delivery of payloads into the arterial wall.

Liposomes have a number of unique characteristics that are particularly suited for payload delivery (Huang 2002). The compartments of aqueous core and lipid bilayer can carry both hydrophilic and lipophilic drugs, respectively. The surface of liposomes can be modified to prolong their in vivo circulation or functionalized to display homing ligands for specific targeting (Klegerman 2002). Echogenic liposomes have the added feature of “air pockets” within the lipid bilayer that allow their visualization by ultrasound in molecular imaging (Kee 2014; Hamilton 2004; Huang 2002), serve as a reservoir for bioactive gases such as nitric oxide (Huang 2009), or respond to ultrasound-triggered sonoporation for controlled drug release (Huang 2002).

Nitric oxide and pioglitazone were chosen for their synergistic effects on restoring endothelial function, inhibiting inflammatory cellular recruitment, and reducing neointimal hyperplasia. Nitric oxide production is impaired in damaged endothelium after angioplasty and is responsible for de novo and peri- and/or in-stent restenosis (Myers 1996; Piatti 2003). Stents eluting anti-proliferative agents such as sirolimus and paclitaxel are more deleterious than bare metal stents in impairing endothelium dependent relaxation responses to acetylcholine, reducing endothelial nitric oxide synthase expression and local nitric oxide production (Togni 2007; Shin 2007). These lead to a failure of re-endothelialization and vascular repair and account for the high rates of very late stent thrombosis in association with drug-eluting stent implantation (Nakazawa 2009). Incorporation and local release of nitric oxide-releasing substances via stent struts may reduce platelet adhesion and intimal hyperplasia but do not solve the issues of long-term exposure to stent components. One option to restore local nitric oxide production could be achieved by increasing local nitric oxide synthase activity. Adenovirus-mediated nitric oxide synthase gene transfer restored nitric oxide production and reduced neointima formation after angioplasty (Janssens 1998; Varenne 1998; von der Thusen 2004). However, longevity of gene expression and toxic effects of vector-induced inflammation are the main concerns of gene transfer vector technology. Direct delivery of nitric oxide to the arterial wall is an attractive approach as this does not reduce eNOS uncoupling-dependent reactive oxygen species generation. The effective dose of NO delivery to the arterial wall can be tuned by varying the amount and rate of NO-ELIP infusion and adjusting the ultrasound parameters to control payload release. However, the acute effect of nitric oxide is short-lived and an additional agent is necessary to sustain an environment for promoting re-endothelialization and reducing local inflammation. PPARγ agonists such as pioglitazone exert their pleiotropic effects at a gene level via their binding to peroxisome proliferator response element. PPARγ activation plays an important role in promoting endothelial proliferation by reducing the production of plasminogen activator inhibitor type 1 (PAI-1) (Hong 2003), reducing leukocyte infiltration by suppressing adhesion molecule expression in the endothelial cells (Wang 2002; Jackson 1999), inhibiting vascular smooth muscle migration by controlling the mitogen-activated protein kinase (MAPK) pathway (Goetze 1999; Ricote 1998), and reducing inflammation by attenuating cytokine production and nuclear factor-KB transcription activity (Jackson 1999; Goetze 1999; Ricote 1998; Jiang 1998).

We have demonstrated that echogenic liposomes containing both nitric oxide and rosiglitazone could attenuate intimal proliferation in balloon-injured carotid arteries in rabbits (Moody 2016) and that echogenic liposomes containing both nitric oxide and pioglitazone could attenuate intimal proliferation in balloon-injured arteries in miniswine (Moody 2017). In the current study, we have improved the efficiency of ELIP-mediated payload delivery by active targeting with anti-ICAM-1 antibody and ultrasound-activated payload release with the EKOS catheter.

In conclusion, a combined strategy of active targeting and ultrasound-facilitated delivery of nitric oxide and pioglitazone was effective in attenuating neointimal growth and in-stent restenosis in stented peripheral arteries. This sets the stage for further development of the drug delivery ultrasound catheter for direct therapy in various vascular territories.

### Example 2: First Liposomes Comprising NO and Second Liposomes Comprising Rosiglitazone

Composition

Combination product consisting of freeze-dried echogenic liposomes (ELIP; contrast agent) conjugated to a monoclonal antibody (biologic) specific for human intercellular adhesion molecule-1 (ICAM-1) and loaded with the anti-inflammatory thiazolidinedione drug rosiglitazone (RGN; small drug molecule).

Preparation of Composition

Therapeutic ELIP preparation: Liposomes (phospholipid bilayer vesicles enclosing an aqueous space) are novel agents that permit evaluation of vasoactive and pathologic atheroma/endothelium. The composition of ELIP is egg phosphatidylcholine/dipalmitoylphosphatidylcholine/1,2-dipalmitoyl-sn-glycero-3-phosphoethanolamine-N-[methoxy(polyethyleneglycol)-2000]/dipalmitoylphosphatidylglycerol/cholesterol (27:42:8:8:15, molar percent). Lipid components for each sample (measured in mole %) are mixed in a 4 ml glass vial as chloroform solutions. The chloroform is then removed by evaporation under argon while the vial is rotated in a 50° C. water bath. The resulting lipid film is then placed under vacuum for 4 h at <100 mTorr pressure for complete removal of the solvent. The dry lipid film is rehydrated with deionized water at 10 mg of lipid/ml. For all experimental compositions (excluding the standard preparation), the hydrated lipid is then incubated at 55° C. for 30 min to insure all lipids are in the liquid crystalline phase during hydration. The mixture is then sonicated in a water bath for 5 min, following which an equal volume of 0.4 M mannitol is then added to give a final mannitol concentration of 0.2 M. Samples of 5 mg are then frozen on dry ice and lyophilize for 24 h.

Once lyophilized MPB-ELIP have been prepared, thiolated MAb (enlimomab)-involving SPDP activation and OTT reduction-is conjugated via a thioether linkage overnight with stirring at room temperature. Unconjugated protein and small molecule reactants are removed by chromatography on a Sepharose CL-4B column.

NO-ELIP preparation: For the in vivo study, liposomes were composed of 1,2-dipalmitoyl-snglycero-3-phosphocholine (Avanti Polar Lipids), 1,2-dioleoyl-sn-glycero-3-phosphocholine, and cholesterol at a molar ratio of 60:30:10 with the addition of 6% (molar ratio) carbonylmethoxypolyethyleneglycol-2000-2-dipalmitoyl-sn-glycero-3-phosphoethanolamine (DPPE-PEG2000). Addition of PEG-phosphoethanolamine improves circulation time. In a previous study, we have shown intravenous injection of this ELIP formulation with ultrasound release over the carotid artery resulted in an enhanced effect using another bioactive gas encapsulated ELIP. Lipids (5 mg) were mixed in chloroform and vaporized with argon in a 50° C. water bath to form a thin film on a glass vial wall. The lipid film was placed under high vacuum (<100 Torr) for 4-6 hours to remove the solvent completely. The dry lipid film is rehydrated with deionized water at 10 mg of lipid/mL. The hydrated lipid is then incubated at 55° C. for 30 min to insure all lipids are in the liquid crystalline phase during hydration. The mixture is then sonicated in a water bath for 5 min, following which an equal volume of 0.32 M mannitol is then added. Samples of 5 mg were transferred to a 2 mL glass vial and frozen on dry ice (−80° C.) for 4 hours. The frozen sample was then lyophilized for 48 hrs. After lyophilization, the dry cake is topped with inner gas, organ, and capped with a rubber septum. To make NO-ELIP, a mixture (10 mL) of NO (Specialty Gases of America Inc., Toledo, Ohio, USA) and octafluoropropane or argon (Matheson Tri-Gas, Houston, Tex., USA) at a ratio of 1:9 was deoxygenated by bubbling through 5 M NaOH. The deoxygenated mixture of NO and octafluoropropane or argon was injected into the lyophilized dry cake via rubber septum using a 12 mL syringe attached to a 27 G×0.5 inch needle. The dry cake was reconstituted with deoxygenated water containing NO and octafluoropropane or argon (1:9).

Rosiglitazone-ELIP preparation: The ELIP for drug delivery will be composed of mixtures of phosphatidylcholine, phosphatidylglycerol (PG), cholesterol (CH), and at least for all preparations to be conjugated, a protein-reactive derivative of phosphatidylethanolamine (PE) will be used. Rosiglitazone will first be complexed with hydroxypropyl-beta-cyclodextrin (HPβCD) to improve drug solubility (by a magnitude of 700-fold) (Huang 2007). The inclusion complex of rosiglitazone with HPl3CD derivative will be prepared by the freeze-drying method. HPβCD (0.08 mmol) will be dissolved in 5 ml 0.32 M mannitol and the solution will be added dropwise in rosiglitazone powder. The mixture will be allowed to stir in the absence of light at room temperature and then centrifuged at 15,800 g for 30 min. Rosiglitazone-cyclodextrin complex and rosiglitazone without cyclodextrin will then be added to the lipid dispersion during ELIP formulation using the dehydration-rehydration-lyophilization.

Results

We have demonstrated the ability of our MAb-conjugated ELIP to effectively localize to vascular targets expressing fibrin, ICAM-1, VCAM-1, αᵥβ₃ integrin, and tissue factor in mouse, rabbit and miniswine atherosclerosis models (Lanza U.S. Pat. No. 5,612,057; Holland U.S. Pat. No. 7,300,414; Holland PCT patent application; Holland U.S. Ser. No. 13/409,634; Huang WO 2014/0261172). We observed enhancement of IVUS and TVUS (transvascular ultrasound) imaging ranging from 8 to 40% after local, intraarterial administration of targeted ELIP. The Yucatan miniswine induced atheroma model of atherosclerosis is generally accepted as one of the two animal models (along with the cynamologous monkey) most similar to the pathogenesis of human atherosclerosis for preclinical translational research.

NO-ELIP: Coencapsulation of NO with OFP enabled the adjustment the amount of encapsulated NO. A total of 10 μl of gas can be encapsulated into 1 mg liposomes. The release profile of NO from NO-ELIP demonstrated an initial rapid release followed by a slower release over 8 hours. Sixty-eight percent of cells remained viable when incubated with 80 μg/ml of NO/Ar-ELIP for 4 hours. NO delivery to VSMC using NO/Ar-ELIP was 7-fold higher than unencapsulated NO. NO/Ar-ELIP remained effective NO delivery to VSMC even in the presence of hemoglobin. Local NO-ELIP administration to balloon-injured carotid arteries attenuated the development of intimal hyperplasia and reduced arterial wall thickening by 41±9%.

Rosiglitazone-ELIP: Acute arterial injury model created in rabbits by balloon injury to the common carotid artery has been used. NO and rosiglitazone loaded ELIP were administered intraarterially into the common carotid artery. Fourteen days later the carotid arteries were removed for histological examination of intimal thickening. Delivery of rosiglitazone or NO/argon loaded ELIP intraarterially resulted in 75+/−13% or 51+/−6% inhibition of intimal proliferation. With NO and rosiglitazone coencapsulation inhibition was reached 88+/−15%. These results demonstrated that delivery of NO and rosiglitazone co-encapsulated ELIP is a promising method for the treatment of atherosclerosis.

Discussion

Our treatment scheme, i.e., administration of an anti-inflammatory drug in a targeted, controlled release formulation locally to the stented atheroma immediately post-PCI, is expected to markedly decrease neoatherosclerosis formation that occurs in 16% of bare metal stents and 31% of drug-eluting stents (Yahagi 2016). In addition, bathing the persistent atheroma with anti-inflammatory agents while maintaining vascular reactivity with vasodilators can effectively stabilize the persistent atheroma and mitigate many processes that would require long-term anticoagulant/anti-platelet therapy in patients who are not candidates for stents (those requiring surgery soon after PCI, those who cannot take anticoagulants, stroke patients who are fall risks, etc.) This neoatherosclerosis formation largely accounts for late-stage thrombosis in stented atheroma (Yahagi 2016); thus, removal of this likelihood should obviate the need for chronic anticoagulant therapy.

### Example 3: First Liposomes Comprising NO and Second Liposomes Comprising Pioglitazone

Compositions

Combination product consisting of freeze-dried echogenic liposomes (ELIP; contrast agent) conjugated to a peptide specific for fibrin and loaded with the anti-inflammatory thiazolidinedione drug pioglitazone (PGN; small drug molecule).

Preparation of Compositions

Pioglitazone-ELIP preparation: Liposomes (phospholipid bilayer vesicles enclosing an aqueous space) are novel agents that permit evaluation of vasoactive and pathologic atheroma/endothelium. The composition of ELIP is: 1,2-distearoyl-sn-glycero-3-phosphocholine (DSPC); 1,2-dioleoyl-sn-glycero-3-phosphoethanolamine-N-[4-(p-maleimidophenyl)butyramide] (MPBDOPE); 1,2-dioleoyl-sn-glycero-3-phosphocholine (DOPC); dipalmitoylphosphatidylglycerol (DPPG), and cholesterol. Lipid components and pioglitazone for each sample (measured in mole %) are mixed in a 4 ml glass vial as chloroform solutions. The chloroform is then removed by evaporation under argon while the vial is rotated in a 50° C. water bath. The resulting lipid film is then placed under vacuum for overnight at <100 mTorr pressure for complete removal of the solvent. The dry lipid film is rehydrated with deionized water containing 0.32 M mannitol at 10 mg of lipid/ml. For all experimental compositions (excluding the standard preparation), the hydrated lipid is then incubated at 55° C. for 30 min to insure all lipids are in the liquid crystalline phase during hydration. The mixture is then sonicated in a water bath for 5 min. Samples of 5 mg are then frozen on dry ice and lyophilized for 48 hrs. After lyophilization, the dry cake is topped with octafluoropentane gas.

Peptide) Once frozen or lyophilized pioglitazone-loaded MPB-ELIP have been prepared, the peptide, gly-pro-arg-pro-pro-gly-gly-gly-cys (GPRPPGGGC), (SEQ ID NO:1) is conjugated via a thioether linkage overnight with stirring at room temperature. The amino-terminal pentapeptide has been shown to bind to fibrin, which is also known to be a marker for late-stage atheroma. Conjugation of liposomal 1,2-dioleoyl-sn-glycero-3-phosphoethanolamine-N-[4-(p-maleimidophenyl)butyramide] (MPB-DOPE) to the carboxy-terminal cysteinyl thiol group (separated from the fibrin-binding moiety by a triglycyl spacer) through a thioether linkage is performed after pioglitazone loading. Unconjugated peptide is removed by centrifugation at 10,000×g for 10 minutes, followed by two centrifugal washes with 0.32 M mannitol.

NO-ELIP preparation: For the in vivo study, liposomes were composed of egg phosphatidylcholine/dipalmitoylphosphatidylcholine/, 2-dipalmitoyl-sn-glycero-3-phosphoethanolamine-N-[methoxy(polyethyleneglycol)-2000]/dipalmitoylphosphatidylglycerol/cholesterol (27:42:8:8:15, molar percent). In a previous study, we have shown intravenous injection of this ELIP formulation with ultrasound release over the carotid artery resulted in an enhanced effect using another bioactive gas encapsulated ELIP. Lipids (5 mg) were mixed in chloroform and vaporized with argon in a 50° C. water bath to form a thin film on a glass vial wall. The lipid film was placed under high vacuum (<100 Torr) for 4-6 hours to remove the solvent completely. The dried lipid film was hydrated with deionized water and sonicated for 5 minutes, following which an equal volume of 0.32 M mannitol is then added. Samples of 5 mg lipids are then frozen on dry ice and lyophilize for 24 h. After lyophilization, the dry cake is vacuumed to de-oxygenate and topped with octafluoropentane or argon gas. Before use, the sample is reconstituted with NO/Octafluoropentane (1:9) or NO/argon (1:9) containing water to form NO-ELIP.

NO/Octafluoropentane water preparation. Deionized water (2 ml) is added in glass vial capped with a Teflon rubber septum. A mixture (10 ml) of NO (Specialty Gases of America Inc., Toledo, Ohio, USA) and Octafluoropentane (Matheson Tri-Gas, Houston, Tex., USA) at a ratio of 1:9 was purified by bubbling through deoxygenated 5 M NaOH to remove oxygen.25 The deoxygenated gaseous mixture of NO and octafluoropentane is injected into the degassed water in the glass vial through a Teflon rubber septum using a 4 ml syringe attached to a 27 G×0.5 inch needle. The pressurized water is stored at 4° C. refrigerator for at least 24 hours.

Results

Atherosclerosis was induced in Yucatan miniswines by a standard technique of cholesterol feeding and balloon denudation. At 2 weeks after the initiation of the hyperlipidemic diet, the right carotid and both subclavian arteries were subjected to balloon denudation under fluoroscopic guidance. Six to eight weeks after balloon denudation, neointimal hyperplasia was well developed in the intervened arteries and was assessed by IVUS imaging. Then, balloon angioplasty and bare metal stents were deployed in all the denuded arteries. The stented arteries were randomized to receive a combination of NO-ELIP and anti-ICAM-1-PGN-ELIP with or without ultrasound application. The ELIPs were introduced to the stented arteries via the EKOS catheter. At the same time, therapeutic ultrasound was delivered using the EKOS EndoSonic Endovascular system with the catheter tip placed just proximal to the stented arteries and the ultrasound elements within the stented segment. Four weeks after therapeutic interventions, IVUS imaging was repeated in the stented vessels prior to euthanasia. Arteries were harvested with the stents in situ and processed for histological staining. The extent of ISR was evaluated by measuring the intima: media ratio in the histological sections and the atheroma volume in the IVUS images. In the stented segments treated with ELIP without ultrasound triggered release, neointimal formation was visualized beyond the stents. However, in the stented segments treated with both ELIP and ultrasound, the formation of neointima was attenuated. Quantitatively, ELIP and ultrasound treatment reduced the intima:media ratio by more than 40%. This suggests that ultrasound treatment enhances the biological effects of the ELIP payload and reduced neointimal formation in the stented arteries. IVUS imaging also confirmed a similar observation, showing a reduction in the thickness of the neointima in the arteries treated with both ELIP and ultrasound. The ex-vivo evaluation of neointimal formation by histological analysis agreed with that by IVUS.

Discussion

Our treatment scheme, i.e., administration of an anti-inflammatory drug in a targeted, controlled release formulation locally to the stented atheroma immediately post-PCI, is expected to markedly decrease neoatherosclerosis formation that occurs in 16% of bare metal stents and 31% of drug-eluting stents (Yahagi 2016). In addition, bathing the persistent atheroma with anti-inflammatory agents while maintaining vascular reactivity with vasodilators can effectively stabilize peri- and/or in-stent atheroma and mitigate many processes that would require long-term anticoagulant/anti-platelet therapy in patients who are not candidates for stents (those requiring surgery soon after PCI, those who cannot take anticoagulants, stroke patients who are fall risks, etc.). This neoatherosclerosis formation largely accounts for late-stage thrombosis in stented atheroma (Yahagi 2016); thus, removal of this likelihood should obviate the need for chronic antiplatelet therapy.

### Example 4: Stability and Shelf Life of First Liposomes Comprising NO and Second Liposomes Comprising Pioglitazone

We prepared eleven batches of fibrin-binding peptide (PAFb)-conjugated PGN-loaded ELIP. The results, in terms of conjugation efficiency and fibrin-binding affinity, are summarized in Table 1.

TABLE 1
Composite Conjugation and Targeting Data
for 11 PAFb-PGN-ELIP batches
Mean ± SE

| | |
|---|---|
| Conjugation Efficiency | Fibrin-Binding |

| μg peptide/ | No. molecules/ | Affinity (K_{D}) |
|---|---|---|
| mg lipid | liposome | μM |
| 1.63 | 2.11 ± 0.50 × 10⁶ | 1.72 ± 0.11 |

Because ELIP-conjugated peptide cannot be assessed directly, we developed a quantitative inhibition ELISA in which inhibition of biotinylated peptide binding to immobilized fibrinogen is determined, to measure conjugation efficiency (CE). We validated this assay with a tetramethylrhodamine isothiocyanate (TRITC)-labeled peptide conjugated to the ELIP. For preparation of double fluorescently labeled PAFb-OFP-PGN-ELIP, where the Pioglitazone (PGN) is labeled with fluorescein isothiocyanate (FITC) and the ELIP is labeled with rhodamine, we coupled the FITC to the PGN through an L-cysteinyl bridge, in which the FITC is first conjugated to the cysteine α-amino function and the PGN is coupled to the L-cys FITC through a thioether linkage. The construct is separated from reactant by dialysis through Spectrum CE tubing, MWCO 500-1,000 Da. We found 20-56% recovery of FITC per molecule PGN. The FITC-PGN was loaded into rhodamine-labeled ELIP for in vivo demonstration of PAFb-OFP-PGN-ELIP localization to stented atheroma as described elsewhere herein.

### Example 5: Delivery of First Liposomes Comprising NO and Second Liposomes Comprising Pioglitazone into Arterial Wall with Inhibition of in-Stent Restenosis in Porcine Model

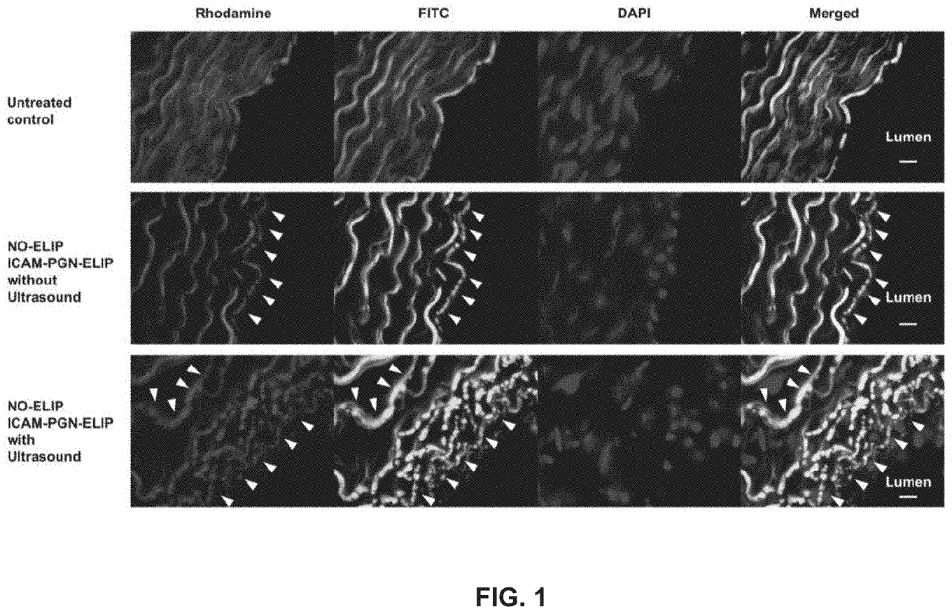

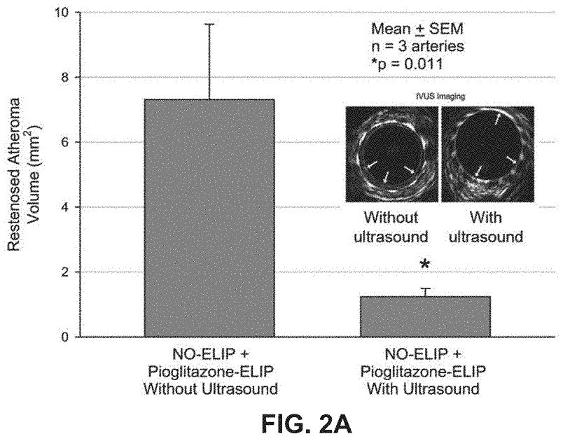

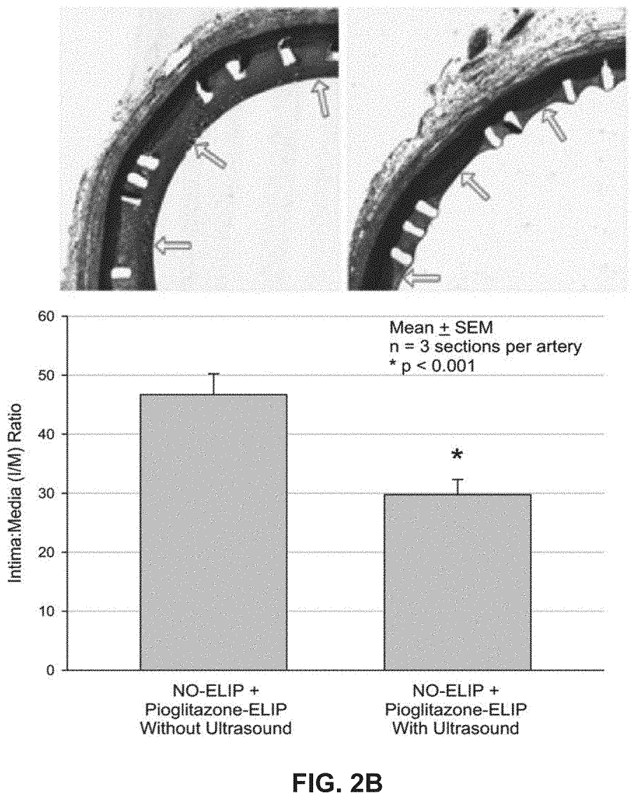

Bare metal stents were implanted into peripheral arteries in cholesterol-fed Yucatan miniswine, followed by direct intra-arterial infusion of NO-ELIP and anti-ICAM1-OFP-PGN-ELIP exposed to an EKOS EndoSonic Endovascular catheter and ultrasonic crystals with or without ultrasound activation. The extent of in-stent restenosis at baseline and 4 weeks after stent implantation was quantified using IVUS imaging in vivo and histological analysis. In the absence of EKOS ultrasound, anti-ICAM1-OFP-PGN-ELIP were seen localized to the endothelial surface of the arterial wall (FIG. 1, middle panel). With EKOS ultrasound, fluorescently-labeled ELIP were shown to penetrate to at least 60 μm into the intima and media of the arteries (FIG. 1, bottom panel). Ultrasound-facilitated delivery of ELIP formulations into stented peripheral arteries attenuated the development of neointimal volume from 7.31±2.32 mm3 (without ultrasound) to 1.24±0.25 mm3 (with ultrasound) (FIG. 2A). Histological analysis revealed a reduction in intima media ratio from 46.71±3.52 (without ultrasound) to 29.76±2.56 (FIG. 2B). The EKOS endovascular delivery system facilitated site-specific intra-arterial drug delivery and enhanced therapeutic release into stented arteries.

### Example 6: Pharmacokinetic Studies of ELIP

We completed preliminary pharmacokinetic studies for ELIP, utilizing a quantitative bioanalytical assay for rhodamine-labeled ELIP, in miniswine in conjunction with a localization study. Results indicated that the arterially administered ELIP washed into the systematic circulation 5 minutes after completion of the infusion and were gone from the venous circulation at 10 minutes.

All of the compositions and methods disclosed and claimed herein can be made and used without undue experimentation in light of the present disclosure. While the compositions and methods of this disclosure have been described in terms of particular embodiments, it will be apparent to those of skill in the art that variations may be applied to the compositions and methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

### REFERENCES

The following references, to the extent that they provide exemplary procedural or other details supplementary to those set forth herein, are specifically incorporated herein by reference.
Abdulhannan P, Russell D A and Homer-Vanniasinkam S. Peripheral arterial disease: a literature review. Br Med Bull. 2012; 104:21-39.
Alfonso F, Byrne R A, Rivero F, Kastrati A. Current treatment of in-stent restenosis. Journal of the American College of Cardiology. 2014; 63(24):2659-73. Epub 2014/03/19. doi:10.1016/j.jacc.2014.02.545. PubMed PMID: 24632282.
Beckman J, Raji A, Plutzky J. Peroxisome proliferator activated receptor gamma and its activation in the treatment of insulin resistance and atherosclerosis: issues and opportunities. Curr Opin Cardiol.
Bekeredjian R, Chen S, Frenkel P A, Grayburn P A, Shohet R V. Ultrasound-targeted microbubble destruction can repeatedly direct highly specific plasmid expression to the heart. Circulation. 2003; 108(8):1022-6.
Brower V. Stents and biology combination for restenosis. Nat Biotechnol. 1996; 14:422.
Buchanan K D, Huang S L, Kim H, McPherson D D, MacDonald R C. Encapsulation of NF-kappaB decoy oligonucleotides within echogenic liposomes and ultrasound-triggered release. J Control Release. 2010; 141(2):193-8.
Chen H, Brayman A A, Evan A P, Matula T J. Preliminary observations on the spatial correlation between short-burst microbubble oscillations and vascular bioeffects. Ultrasound Med Biol. 2012; 38(12):2151-62.
Chen S, Shohet R V, Bekeredjian R, Frenkel P, Grayburn P A. Optimization of ultrasound parameters for cardiac gene delivery of adenoviral or plasmid deoxyribonucleic acid by ultrasound-targeted microbubble destruction. J Am Coll Cardiol. 2003; 42(2):301-8.
Cheng J Y, Shaw G J, Holland C K. In vitro microscopic imaging of enhanced thrombolysis with 120-kHz ultrasound in a human clot model. Acoust Res Lett Online. 2005; 6(1):25-9.
Coussios C C, Holland C K, Jakubowska L, Huang S L, MacDonald R C, Nagaraj A, et al. In vitro characterization of liposomes and Optison by acoustic scattering at 3.5 MHz. Ultrasound Med Biol. 2004; 30(2):181-90.
Datta S, Coussios C C, Ammi A Y, Mast T D, de Courten-Myers G M, Holland C K. Ultrasound-enhanced thrombolysis using Definity as a cavitation nucleation agent. Ultrasound Med Biol. 2008; 34(9):1421-33.
Datta S, Coussios C C, McAdory L E, Tan J, Porter T, De Courten-Myers G, et al. Correlation of cavitation with ultrasound enhancement of thrombolysis. Ultrasound Med Biol. 2006; 32(8): 1257-67.
Duda S H, Bosiers M, Lammer J, Scheinert D, Zeller T, Tielbeek A, Anderson J, Wiesinger B, Tepe G, Lansky A, Mudde C, Tielemans H and Beregi J P. Sirolimus eluting versus bare nitinol stent for obstructive superficial femoral artery disease: the SIROCCO II trial. J Vase lnterv Radial. 2005; 16:331-8.
Duda S H, Pusich B, Richter G, Landwehr P, Oliva V L, Tielbeek A, Wiesinger B, Hak J B, Tielemans H, Ziemer G, Cristea E, Lansky A and Beregi J P. Sirolimus—eluting stents for the treatment of obstructive superficial femoral artery disease: six-month results. Circulation. 2002; 106:1505-9.
Everbach E C, Francis C W. Cavitational mechanisms in ultrasound-accelerated thrombolysis at 1 MHz. Ultrasound Med Biol. 2000; 26(7):1153-60.
Farb A, Weber D K, Kolodgie F D, Burke A P and Virmani R. Morphological predictors of restenosis after coronary stenting in humans. Circulation. 2002; 105:2974-80.
Farny C H, Holt R G, Roy R A. Temporal and spatial detection of HIFU-induced inertial and hot-vapor cavitation with a diagnostic ultrasound system. Ultrasound Med Biol. 2009; 35(4):603-15.
Finn A V, Nakazawa G, Joner M, Kolodgie F D, Mont E K, Gold H K and Virmani R. Vascular responses to drug eluting stents: importance of delayed healing. Arterioscler Thromb Vase Biol. 2007; 27:1500-10.
Goetze S, Xi X P, Kawano H, Gotlibowski T, Fleck E, Hsueh W A and Law R E. PPAR gamma-ligands inhibit migration mediated by multiple chemoattractants in vascular smooth muscle cells. J Cardiovasc Pharmacol. 1999; 33:798-806.
Gruber M J, Bader K B, Holland C K. Cavitation thresholds of contrast agents in an in vitro human clot model exposed to 120-kHz ultrasound. J Acoust Soc Am. 2014; 135(2):646-53.
Gyongy M, Coussios C C. Passive spatial mapping of inertial cavitation during HIFU exposure. IEEE Trans Biomed Eng. 2010; 57(1):48-56.
Hamilton A J, Huang S L, Warnick D, Rabbat M, Kane B, Nagaraj A, Klegerman M and McPherson D D. Intravascular ultrasound molecular imaging of atheroma components in vivo. J Am Coll Cardiol. 2004; 43:453-60.
Haworth K, Salgaonkar V A, Corregan N M, Holland C K, Mast T D. Spatial specificity and sensitivity of passive cavitation imaging for monitoring high-intensity focused ultrasound thermal ablation in ex vivo bovine liver. Proc Meet Acoust. 2013; 19(1):075022.
Haworth K J, Mast T D, Radhakrishnan K, Burgess M T, Kopechek J A, Huang S L, et al. Passive imaging with pulsed ultrasound insonations. J Acoust Soc Am. 2012; 132(1):544-53.
Haworth K J, Raymond J L, Radhakrishnan K, Moody M R, Huang S, Peng T, et al. Trans-stent ultrasound imaging and cavitation detection. Ultrasound Med Biol. 2015: In press.
Hernot S, Klibanov A L. Microbubbles in ultrasound-triggered drug and gene delivery. Adv Drug Deliv Rev. 2008; 60(10):1153-66.
Hitchcock K E, Caudell D N, Sutton J T, Klegerman M E, Vela D, Pyne-Geithman G J, et al. Ultrasound-enhanced delivery of targeted echogenic liposomes in a novel ex vivo mouse aorta model. J Control Release. 2010; 144(3):288-95.
Hitchcock K E, Ivancevich N M, Haworth K J, Caudell Stamper D N, Vela D C, Sutton J T, et al. Ultrasound-enhanced rt-PA thrombolysis in an ex vivo porcine carotid artery model. Ultrasound Med Biol. 2011; 37(8):1240-51.
Holland C K, Datta S, Mast T D, Ivancevich N M, Hitchcock K E, Haworth K J (2010). Ultrasound mediated inducement, detection and enhancement of stable cavitation. PCT Patent application.
Holland C K, Hitchcock K E, Haworth K J, Ivancevich N M, Mast T D (2012). Methods of enhancing delivery of drugs using ultrasonic waves and systems for performing the same. U.S. patent application Ser. No. 13/409,634.
Holland C K, Kanter O S, Busse L J, Wagner K R (2007). Transcranial ultrasound thrombolysis system and method of treating a stroke. U.S. Pat. No. 7,300,414.
Hong H K, Cho Y M, Park K H, Lee C T, Lee H K and Park K S. Peroxisome proliferator-activated receptor gamma mediated inhibition of plasminogen activator inhibitor type 1 production and proliferation of human umbilical vein endothelial cells. Diabetes Res Clin Pract 2003; 62:1-8.
Huang S, Hamilton A J, Tiukinhoy S D, Nagaraj A, Kane B J, Klegerman M, McPherson D D and MacDonald R C. Liposomes as ultrasound imaging contrast agents and as ultrasound-sensitive drug delivery agents. Cell Mol Biol Lett 2002; 7:233-5.
Huang S, McPherson D D, MacDonald R C. A novel pressure/freeze method to form gas and drug coencapsulated liposomes. Am Assoc Pharm Soc. 2006a:W5173.
Huang S, McPherson D D, MacDonald R C. Echogenic liposomes for nitric oxide delivery. Circulation. 2006b; 114: 11-437.
Huang S L, Hamilton A J, Pozharski E, Nagaraj A, Klegerman M E, McPherson D D and MacDonald R C. Physical correlates of the ultrasonic reflectivity of lipid dispersions suitable as diagnostic contrast agents. Ultrasound Med Biol. 2002; 28:339-48.
Huang S L, Kee P H, Kim H, Moody M R, Chrzanowski S M, Macdonald R C and McPherson D D. Nitric oxide-loaded echogenic liposomes for nitric oxide delivery and inhibition of intimal hyperplasia. J Am Coll Cardiol. 2009; 54:652-9.
Huang S L, Klegerman M E, Geng Y J, Kim H, McPherson D D (2014). Neuroprotective liposome compositions and methods for treatment of stroke. WO2014/026117.
Huang S L, MacDonald R C. Acoustically active liposomes for drug encapsulation and ultrasound-triggered release. Biochim Biophys Acta. 2004; 1665(1-2):134-41. PMID: 15471579.
Huang S L, McPherson D D, MacDonald R C. A method to co-encapsulate gas and drugs in liposomes for ultrasound-controlled drug delivery. Ultrasound Med Biol. 2008; 34(8): 1272-80. PMID: 18407399
Jackson S M, Parhami F, Xi X P, Berliner J A, Hsueh W A, Law R E, and Demer L L. Peroxisome proliferator-activated receptor activators target human endothelial cells to inhibit leukocyte-endothelial cell interaction. Arterioscler Thromb Vase Biol. 1999; 19:2094-104.
Janssens S, Flaherty D, Nang Z, Varenne O, van Pelt N, Haustermans C, Zoldhelyi P, Gerard R, and Collen D. Human endothelial nitric oxide synthase gene transfer inhibits vascular smooth muscle cell proliferation and neointima formation after balloon injury in rats. Circulation. 1998; 97:1274-81.
Jiang C, Ting A T, and Seed B. PPAR-gamma agonists inhibit production of monocyte inflammatory cytokines. Nature. 1998; 391:82-6.
Kee P H, Kim H, Huang S, Laing S T, Moody M R, Vela D, Klegerman M E and McPherson D D. Nitric oxide pretreatment enhances atheroma component highlighting in vivo with intercellular adhesion molecule-1-targeted echogenic liposomes. Ultrasound Med Biol. 2014; 40:1167-76.
Kim H, Britton G L, Peng T, Holland C K, McPherson D D and Huang S L. Nitric oxide-loaded echogenic liposomes for treatment of vasospasm following subarachnoid hemorrhage. Int J Nanomedicine. 2014; 9:155-65.
Kim H, Kee P H, Rim Y, Moody M R, Klegerman M E, Vela D, Huang S L, McPherson D D, and Laing S T. Nitric oxide improves molecular imaging of inflammatory atheroma using targeted echogenic immunoliposomes. Atherosclerosis. 2013; 231:252-60.
Kim H, Moody M R, Laing S T, Kee P H, Huang S L, Klegerman M E and McPherson D D. In vivo volumetric intravascular ultrasound visualization of early/inflammatory arterial atheroma using targeted echogenic immunoliposomes. Invest Radiol. 2010; 45:685-91.
Kim S, Kim D J, Geyer M A, Howell S B. Multivesicular liposomes containing 1-beta-Darabinofuranosylcytosine for slow-release intrathecal therapy. Cancer Res. 1987; 47(15):3935-7.
Klegerman M E, Hamilton A J, Huang S L, Tiukinhoy S D, Khan A A, MacDonald R C, and McPherson D D. Quantitative immunoblot assay for assessment of liposomal antibody conjugation efficiency. Anal Biochem. 2002; 300:46-52.
Klegerman M E, Wassler M, Huang S L, Zou Y, Kim H, Shelat H S, et al. Liposomal modular complexes for simultaneous targeted delivery of bioactive gasses and therapeutics. Journal of controlled release: Official journal of the Controlled Release Society. 2010; 142(3); 326-31.
Kodama T, Tomita Y, Koshiyama K, Blomley M J. Transfection effect of microbubbles on cells in superposed ultrasound waves and behavior of cavitation bubble. Ultrasound Med Biol. 2006; 32(6):905-14.
Krishna P D, Shankar P M, Newhouse V L. Subharmonic generation from ultrasonic contrast agents. Phys Med Biol. 1999; 44(3):681-94.
Laing S T, Moody M R, Kim H, Smulevitz B, Huang S L, Holland C K, McPherson D D and Klegerman M E. Thrombolytic efficacy of tissue plasminogen activator-loaded echogenic liposomes in a rabbit thrombus model. Thromb Res. 2012; 130:629-35.
Lammer J, Bosiers M, Zeller T, Schillinger M, Boone E, Zaugg M J, Verta P, Peng L, Gao X, Schwartz L B. First clinical trial of nitinol self-expanding everolimus-eluting stent implantation for peripheral arterial occlusive disease. J Vase Surg. 2011; 54(2):394-401. PMID: 21658885.
Lanza G M, Onyuksel H, Klegerman M E, Vonesh M J, McPherson D D. Acoustically Reflective Liposomes and Methods to Make and Use the Same (1997), U.S. Pat. No. 5,612,057
Lasche J, Weissig V, and Brandl M. Preparation of liposomes. New York: Oxford University Press; 2003.
Lawrie A, Brisken A F, Francis S E, Wyllie D, Kiss-Toth E, Qwarnstrom E E, et al. Ultrasound-enhanced transgene expression in vascular cells is not dependent upon cavitation-induced free radicals. Ultrasound Med Biol. 2003; 29(10):1453-61.
Madanshetty S I, Roy R A, Apfel R E. Acoustic microcavitation: its active and passive acoustic detection. J Acoust Soc Am. 1991; 90(3):1515-26
Moody M R, Huang S, Kim H, Chrzanowski S and McPherson D D. Abstract 5552: Bioactive Gas/Drug Co-encapsulation and Release Improve Attenuation of Intimal Hyperplasmia Following Acute Arterial Injury. Circulation. 2016; 118:S_573-S_573.
Moody M R, Huang S L, Kim H, Chrzanowski S M, McPherson D D. Bioactive gas/drug co-encapsulation and release improves attenuation of intimal hyperplasia following acute arterial injury. Circulation. 2008; 118(18):S573.
Moody, M. R., Huang, S. L., Kim, H., Yin, X., Peng, T., Laing, S. T., Klegerman, M. E., Genstler, C., Haworth, K. J., Holland, C. K., McPherson, D. D., and Kee, P.: Stabilizing peri-stent restenosis using a novel therapeutic carrier. Circulation 136: A 17293, 2017.
Myers P R, Webel R, Thondapu V, Xu X P, Amann J, Tanner M A, Jenkins J S, Pollock J S and Laughlin M R. Restenosis is associated with decreased coronary artery nitric oxide synthase. Int J Cardiol. 1996; 55:183-91.
Nakazawa G, Vorpahl M, Finn A V, Narula J, and Virmani R. One step forward and two steps back with drug-eluting-stents: from preventing restenosis to causing late thrombosis and nouveau atherosclerosis. JACC Cardiovasc Imaging. 2009; 2:625-8.
Newman C M, Bettinger T. Gene therapy progress and prospects: ultrasound for gene transfer. Gene Ther. 2007; 14(6):465-75.
Otto C M. Textbook of clinical echocardiography. Third ed. Philadelphia, Pa.: Elsevier Saunders; 2004.
Peng T, Britton G L, Kim H, Cattano D, Aronowski J, Gretta J, McPherson D D, and Huang S L. Therapeutic time window and dose dependence of xenon delivered via echogenic liposomes for neuroprotection in stroke. CNS Neurosci Ther. 2013; 19:773-84.
Phillips L C, Klibanov A L, Wamhoff B R, Hossack J A. Intravascular ultrasound detection and delivery of molecularly targeted microbubbles for gene delivery. IEEE Trans Ultrason Ferroelectr Freq Control. 2012; 59(7):1596-601.
Piatti P, Di Mario C, Monti L D, Fragasso G, Sgura F, Caumo A, Setola E, Lucotti P, Galluccio E, Ranchi C, Origgi A, Zavaroni I, Margonato A, and Colombo A. Association of insulin resistance, hyperleptinemia, and impaired nitric oxide re lease with in-stent restenosis in patients undergoing coronary stenting. Circulation. 2003; 108:2074-81.
Prokop A F, Soltani A, Roy R A. Cavitational Mechanisms in Ultrasound-Accelerated Fibrinolysis. Ultrasound in Medicine and Biology. 2007; 33(6):924-33.
Ricote M, Li A C, Willson T M, Kelly C J, and Glass C K. The peroxisome proliferator-activated receptor-gamma is a negative regulator of macrophage activation. Nature. 1998; 391:79-82.
Roy R A, Madanshetty S I, Apfel R E. An acoustic backscattering technique for the detection of transient cavitation produced by microsecond pulses of ultrasound. J Acoust Soc Am. 1990; 87(6):2451-8.
Salgaonkar V A, Datta S, Holland C K, Mast T D. Passive cavitation imaging with ultrasound arrays. J Acoust Soc Am. 2009; 126(6):3071-83.
Shankar P M, Dala Krishna P, Newhouse V L. Advantages of subharmonic over second harmonic backscatter for contrast-to-tissue echo enhancement. Ultrasound Med Biol. 1998; 24(3):395-9.
Shi W T, Forsberg F, Hall A L, Chiao R Y, Liu J B, Miller S, et al. Subharmonic imaging with microbubble contrast agents: initial results. Ultrason Imaging. 1999; 21(2):79-94.
Shin D I, Kim P J, Seung K B, Kim D B, Kim M J, Chang K, Lim S M, Jeon O S, Chung W S, Baek S H and Lee M Y. Drug-eluting stent implantation could be associated with long-term coronary endothelial dysfunction. Int Heart J 2007; 48:553-67.
Suzuki J, Ogawa M, Takayama K, Taniyama Y, Morishita R, Hirata Y, et al. Ultrasound-microbubble-mediated intercellular adhesion molecule-1 small interfering ribonucleic acid transfection attenuates neointimal formation after arterial injury in mice. J Am Coll Cardiol. 2010; 55(9):904-13.
Suzuki R, Takizawa T, Negishi Y, Hagisawa K, Tanaka K, Sawamura K, et al. Gene delivery by combination of novel liposomal bubbles with perfluoropropane and ultrasound. J Control Release. 2007; 117(1):130-6.
Tellez A, Krueger C G, Seifert P, Winsor-Hines D, Piedrahita C, Cheng Y, Milewski K, Aboodi M S, Yi G, McGregor J C, Crenshaw T, Reed J D, Huibregtse B, Kaluza G L and Granada J F. Coronary bare metal stent implantation in homozygous LDL receptor deficient swine induces a neointimal formation pattern similar to humans. Atherosclerosis. 2010; 213:518-24.
Togni M, Raber L, Cocchia R, Wenaweser P, Cook S, Windecker S, Meier B, and Hess O M. Local vascular dysfunction after coronary paclitaxel-eluting stent implantation. Int J Cardiol. 2007; 120:212-20.
Unger E C, Hersh E, Vannan M, Matsunaga T O, McCreery T. Local drug and gene delivery through microbubbles. Prog Cardiovasc Dis. 2001; 44(1):45-54.
Unger E C, Hersh E, Vannan M, McCreery T. Gene delivery using ultrasound contrast agents. Echocardiography. 2001; 18(4):355-61.
Varenne O, Pislaru S, Gillijns H, Van Pelt N, Gerard R D, Zoldhelyi P, Van de Werf F, Collen D and Janssens S P. Local adenovirus-mediated transfer of human endothelial nitric oxide synthase reduces luminal narrowing after coronary angioplasty in pigs. Circulation. 1998; 98:919-26.
von der Thusen J H, Fekkes M L, Passier R, van Zonneveld A J, Mainfroid V, van Berkel T J, and Biessen E A. Adenoviral transfer of endothelial nitric oxide synthase attenuates lesion formation in a novel murine model of postangioplasty restenosis. Arterioscler Thromb Vase Biol. 2004; 24:357-62.
Wang N, Verna L, Chen N G, Chen J, Li H, Forman B M, and Stemerman M B. Constitutive activation of peroxisome proliferator-activated receptor-gamma suppresses pro-inflammatory adhesion molecules in human vascular endothelial cells. J Biol Chem. 2002; 277:34176-81.
Yahagi K, Kolodgie F D, Otsuka F, Finn A V, Davis H R, Joner M, et al. Pathophysiology of native coronary, vein graft, and in-stent atherosclerosis. Nature reviews Cardiology. 2016; 13(2):79-98. Epub 2015/10/28. doi: 10.1038/nrcardio.2015.164. PubMed PMID: 26503410.
Patrick H Kee, Melanie R Moody, Shao-Ling Huang, Hyunggun Kim, Xing Yin, Tao Peng, Susan T Laing, Melvin E Klegerman, Deborah Vela, Curtis Genstler, Kevin J Haworth, Christy K Holland, and David D McPherson. Stabilizing Peri-stent Restenosis Using A Novel Therapeutic Carrier. Submitted to JACC Bench to Translational Science, 2019 (Submitted).
Klegerman, M E. Quantitative characterization of targeted nanoparticulate formulations for prediction of clinical efficacy. In Grumezescu A. M. (ed.); Nanoscale Fabrication, Optimization, Scale-up and Biological Aspects of Pharmaceutical Nanotechnology, Chap. 10. Elsevier, Cambridge, Mass., 2018.
K. J. Haworth, A. S. Hannah, H. Shekhar, P. Arunkumar, S-L. Huang, T. Peng, M. Vranish, D. McPherson, C. Genstler, C. K. Holland. “Cavitation activity of pioglitazone echogenic liposomes using an EkoSonic® catheter.” 18^{th} International symposium on Therapeutic Ultrasound (May 15, 2018).
Huang S. L., Klegerman M. E., Peng T., Moody M. R., Holland C. K., Haworth K. J., Genstler C., Hannah A., Levengood S., McPherson D. D., Laing S. T., and Kee P. H. Targeted ultrasound responsive liposomes for therapeutic delivery of anti-inflammatory drugs into the atherosclerotic arterial wall. Circulation 138: A16014, 2018.
M. LaFond, H. Shekhar, N. Salido, K. J. Haworth, A. Hannah, C. Genstler, C. K. Holland. “Cavitation nucleation by Definity® infused through an EkoSonic® catheter.” 19^{th} International symposium on Therapeutic Ultrasound (Jun. 13, 2019).
N. G. Salido, K. J. Haworth, M. Lafond, C. Genstler, C. K. Holland. “Frequency-domain passive cavitation imaging with circular coherence factor.” 19^{th} International symposium on Therapeutic Ultrasound (Jun. 13, 2019).
Yin X., Dandekar M. P., Peng t., Klegerman M. E., Moody M. R., Kim H., McPherson D. D., and Huang S. L. Stroke stabilization following repetitive xenon liposomal delivery. Circulation 138: A17168, 2018.

What is claimed is:

1. A composition, comprising:
a first liposome, comprising at least one lipid selected from the group consisting of saturated phospholipids, unsaturated phospholipids, mixed phospholipids, and cholesterol, and at least one vasodilator; and
a second liposome, comprising at least one lipid selected from the group consisting of saturated phospholipids, unsaturated phospholipids, mixed phospholipids, and cholesterol, at least one thiazolidinedione, and at least one compound having an affinity for at least one component of an atheroma.

2. The composition of claim 1, wherein the at least one thiazolidinedione is selected from the group consisting of rosiglitazone and pioglitazone.

3. The composition of claim 2, wherein the at least one thiazolidinedione is selected from the group consisting of pioglitazone.

4. The composition of claim 1, wherein the at least one compound having an affinity for at least one component of an atheroma is selected from the group consisting of fibrin binding peptides (FBPs) and antibodies to at least one intercellular adhesion molecule (anti-ICAMs).

5. The composition of claim 4, wherein the at least one compound having an affinity for at least one component of an atheroma is selected from the group consisting of an FBP having SEQ ID NO:1.

6. The composition of claim 1, wherein the at least one vasodilator is selected from the group consisting of nitric oxide (NO).

7. The composition of claim 1, wherein the second liposome comprises at least one lipid selected from the group consisting of 1,2-distearoyl-sn-glycero-3-phosphocholine (DSPC); 1,2-dioleoyl-sn-glycero-3-phosphoethanolamine-N-[4-(p-maleimidophenyl)butyramide] (MPB-DOPE); 1,2-dioleoyl-sn-glycero-3-phosphocholine (DOPC); dipalmitoylphosphatidylglycerol (DPPG), and cholesterol.

8. The composition of claim 7, wherein the second liposome further comprises octofluoropentane (OFP).

9. A method, comprising:
administering a first liposome which comprises at least one lipid selected from the group consisting of saturated phospholipids, unsaturated phospholipids, mixed phospholipids, and cholesterol, and at least one vasodilator, into the artery in proximity to the stent;
applying a first ultrasound treatment in proximity to the stent, whereby the at least one vasodilator is released by the first liposome;
administering a second liposome which comprises at least one lipid selected from the group consisting of saturated phospholipids, unsaturated phospholipids, mixed phospholipids, and cholesterol, at least one thiazolidinedione, and at least one compound having an affinity for at least one component of an atheroma, into an artery of a mammal in proximity to a stent;
applying a second ultrasound treatment in proximity to the stent, whereby the at least one thiazolidinedione is absorbed by the atheroma;

10. The method of claim 9, wherein the administering the first liposome is performed prior to the applying the first ultrasound treatment; and the applying the first ultrasound treatment is performed prior to the administering the second liposome.

11. The method of claim 9, wherein the at least one thiazolidinedione is selected from the group consisting of rosiglitazone and pioglitazone.

12. The method of claim 11, wherein the at least one thiazolidinedione is selected from the group consisting of pioglitazone.

13. The method of claim 9, wherein the at least one compound having an affinity for at least one component of an atheroma is selected from the group consisting of fibrin binding peptides (FBPs) and antibodies to at least one intercellular adhesion molecule (anti-ICAMs).

14. The method of claim 13, wherein the at least one compound having an affinity for at least one component of an atheroma is selected from the group consisting of an FBP having SEQ ID NO:1.

15. The method of claim 9, wherein the at least one vasodilator is selected from the group consisting of nitric oxide (NO).

16. The method of claim 9, wherein the second liposome comprises at least one lipid selected from the group consisting of 1,2-distearoyl-sn-glycero-3-phosphocholine (DSPC); 1,2-dioleoyl-sn-glycero-3-phosphoethanolamine-N-[4-(p-maleimidophenyl)butyramide] (MPB-DOPE); 1,2-dioleoyl-sn-glycero-3-phosphocholine (DOPC); dipalmitoylphosphatidylglycerol (DPPG), and cholesterol.

17. The method of claim 9, wherein at least one of the first liposome and the second liposome further comprises octofluoropentane (OFP).

18. A method, comprising:
forming a first liposome, comprising at least one lipid selected from the group consisting of saturated phospholipids, unsaturated phospholipids, mixed phospholipids, and cholesterol;
introducing at least one vasodilator into the first liposome;
combining at least one lipid selected from the group consisting of saturated phospholipids, unsaturated phospholipids, mixed phospholipids, and cholesterol; with at least one thiazolidinedione; in a solvent, to form a second liposome comprising the at least one lipid and the at least one thiazolidinedione; and
conjugating at least one compound having an affinity for at least one component of an atheroma to the second liposome.

19. The method of claim 18, wherein the solvent is selected from the group consisting of chloroform and ethanol.

20. The method of claim 19, wherein the solvent is selected from the group consisting of ethanol.

21. The method of claim 20, wherein the at least one thiazolidinedione is selected from the group consisting of pioglitazone, and the pioglitazone is in the form of pioglitazone-HCl.

22. The method of claim 18, wherein the at least one lipid is selected from the group consisting of 1,2-distearoyl-sn-glycero-3-phosphocholine (DSPC); 1,2-dioleoyl-sn-glycero-3-phosphoethanolamine-N-[4-(p-maleimidophenyl)butyramide] (MPB-DOPE); 1,2-dioleoyl-sn-glycero-3-phosphocholine (DOPC); dipalmitoylphosphatidylglycerol (DPPG), and cholesterol.

23. The method of claim 22, further comprising introducing octofluoropentane (OFP) into at least one of the first liposome and the second liposome.

24. The method of claim 18, wherein the at least one compound having an affinity for at least one component of an atheroma is selected from the group consisting of fibrin binding peptides (FBPs) and antibodies to at least one intercellular adhesion molecule (anti-ICAMs).

25. The method of claim 24, wherein the at least one compound having an affinity for at least one component of an atheroma is selected from the group consisting of an FBP having SEQ ID NO:1, the at least one lipid is selected from the group consisting of MPB-DOPE, and wherein the conjugating the at least one compound having an affinity for at least one component of an atheroma to the second liposome comprises linking the FBP having SEQ ID NO:1 to the MPB-DOPE by a thioether linkage.

26. The method of claim 18, wherein the at least one vasodilator is selected from the group consisting of nitric oxide (NO).


SEQUENCE LISTING (1 sequence)

SEQ ID NO: 1 - protein, 9 aa, Artificial
  Fibrin binding peptide having an affinity for at least one component of an atheroma.
GPRPPGGGC